(19) United States
(12) Patent Application Publication
(10) Pub. No.: US 2011/0182971 A1
(43) Pub. Date: Jul. 28, 2011

(54) TRANSEPIDERMAL DRUG DELIVERY SYSTEM CONTAINING TULOBUTEROL

(76) Inventors: Dong Il Lee, Ansan-si (KR); Mun Seok Han, Suwon-si (KR); Tae Wan Lee, Yongin-si (KR); Jong Kyoo Lee, Seoul (KR); Han Ki Kim, Yongin-si (KR)

(21) Appl. No.: 12/844,538

(22) Filed: Jul. 27, 2010

(30) Foreign Application Priority Data
Jan. 25, 2010 (KR) 10-2010-0006333

(51) Int. Cl. A61F 13/02; A61K 31/135

(52) U.S. Cl. 424/448; 514/653

(57) ABSTRACT

Disclosed is a pharmaceutical composition containing tulobuterol. More specifically, disclosed is a transepidermal drug delivery system including a drug layer containing tulobuterol and a natural rubber-based adhesive material, and a supporter adhered to one surface of the drug layer to support the drug layer, wherein the natural rubber-based adhesive material comprises 10 to 40 parts by weight of a natural rubber, 54.5 to 85 parts by weight of a rosin ester resin and an acid value controller, and the drug layer has a thickness of 25 μm to 75 μm.

This application claims the benefit of Korean Patent Application No. 10-2010-0006333, filed on Jan. 25, 2010, which is hereby incorporated by reference as if fully set forth herein.

### BACKGROUND OF THE INVENTION

1. Field of the Invention

The present invention relates to a pharmaceutical composition comprising tulobuterol. More specifically, the present invention relates to a transepidermal drug delivery system comprising tulobuterol.

2. Discussion of the Related Art

Tulobuterol[2-t-butylamino-1-(2-chlorophenyl)ethanol] is a beta-adrenaline drug which has a secondary amine represented by the following chemical structure 1 and a molecular weight of 227.7, which selectively acts on β2 acceptors of sympathetic nerves and relaxes bronchial smooth muscles. Accordingly, tulobuterol has been used as a bronchodilator to relieve or treat dyspnea of patients who suffer from asthma and respiratory diseases due to respiratory stenosis. Tulobuterol may be provided as a salt.

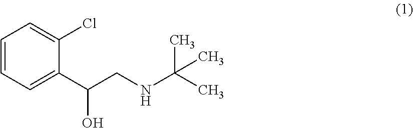

A transepidermal drug delivery system which enables epidermal delivery of a drug can continuously deliver a predetermined amount of the drug to the body for a long time and prevent first-pass metabolism in the liver. In particular, it was known that the transepidermal drug delivery system is a good means to decrease administration frequency of drugs and reduce side effects due to high compliance. Since the skin generally provides considerably low transmission, and most drugs are unsuitable for physical and chemical applications to transepidermal drug delivery systems and are difficult to secure safety to the human body, and drugs in the form of transepidermal drug delivery systems are extremely limited, development of transepidermal drug delivery systems comprising bronchodilators comes into the spotlight. In particular, a great deal of research on transepidermal drug delivery systems comprising tulobuterol is underway.

Japanese Patent Publication No. 7-25669 discloses a transepidermal drug delivery system comprising a high concentration (solubility or higher) of tulobuterol in an adhesive. In accordance with the transepidermal drug delivery system, a high concentration (solubility or higher) of tulobuterol present in the adhesive causes increased drug transepidermal absorption and extended sustained release time. However, over time, tulobuterol is partially dispersed in the form of crystals in the drug layer and tulobuterol crystals are readily generated on the surface of the drug layer. For this reason, the adhesion force of the transepidermal drug delivery system to the skin decreases and thus a predetermined drug release rate cannot be realized.

Korean Patent Nos. 10-0535302, 10-0469995, 10-0579721 and 10-0439659 disclose a matrix-type transepidermal drug delivery system comprising an acrylic adhesive, tulobuterol and a transepidermal absorption accelerator. The acryl adhesive has main monomers, modified monomers and various functional groups such as carboxyl groups, hydroxyl groups, epoxy groups, and organosilane groups. The acrylic adhesives release an excessive amount of drugs early on, although they are varied according to functional groups or monomers, and thus cannot release tulobuterol at a predetermined rate for a long time. In addition, the transepidermal absorption accelerator inevitably imparts even a slight irritation to the skin tissues so that it enables dermal delivery of a drug although it has a composition with superior human safety.

Japanese Patent No. 3930984 discloses a transepidermal drug delivery system wherein tulobuterol is present in a high concentration (5 parts by weight or higher) in an acrylic adhesive or synthetic rubber-based adhesive containing a transepidermal absorption accelerator. The transepidermal drug delivery system disclosed in Japanese Patent No. 3930984 exhibits superior transepidermal absorption, drug efficacy maintenance and superior adhesion. However, the acrylic adhesive may cause excessive drug release early on, and the synthetic rubber-based adhesive may damage the skin, thus causing side effects such as local erythema, pruritus or inflammation.

### SUMMARY OF THE INVENTION

Accordingly, the present invention is directed to a transepidermal drug delivery system containing tulobuterol that substantially obviates one or more problems due to limitations and disadvantages of the related art.

The transepidermal drug delivery system containing tulobuterol should be nontoxic to humans, exhibit high transepidermal absorbance, superior drug efficacy maintenance and high adhesiveness. Accordingly, the core technique of transepidermal drug delivery system is a great deal of pharmaceutical research on the mechanism wherein the transepidermal drug delivery system is composed of ingredients that are non-toxic to the human body and do not damage the skin and a main drug is safely absorbed in the form of a solution. In addition, the transepidermal absorption accelerator inevitably entails skin irritation, although it exhibits superior human safety. There has been an increasing need for transepidermal drug delivery systems containing no transepidermal absorption accelerator, but development of transepidermal drug delivery systems exhibiting the desired drug release rate without any transepidermal absorption accelerator is considerably difficult.

Accordingly, it is one object of the present invention to provide a transepidermal drug delivery system which is nontoxic to the skin, exhibits superior transepidermal absorbance and contains tulobuterol exhibiting superior drug efficacy maintenance and adhesiveness.

It is another object of the present invention to provide a transepidermal drug delivery system containing tulobuterol wherein tulobuterol is safely absorbed in the form of a solution into the skin without containing any transepidermal absorbing agent.

To achieve the object and other advantages and in accordance with the purpose of the invention, as embodied and broadly described herein, provided is a transepidermal drug delivery system comprising a drug layer containing tulobuterol and a natural rubber-based adhesive material and a supporter adhered to one surface of the drug layer to support the drug layer, wherein the natural rubber-based adhesive material comprises 10 to 40 parts by weight of a natural rubber, 54.5 to 85 parts by weight of a rosin ester resin and an acid value controller, and the drug layer has a thickness of 25 μm to 75 μm.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and along with the description serve to explain the principle of the invention. In the drawings:

FIG. 1 is a sectional view illustrating a patch-type transepidermal drug delivery system according to one embodiment of the present invention;

FIG. 2 is a plan view illustrating a release film or a release paper of FIG. 1;

FIG. 3 is a sectional view illustrating a fixer of FIG. 1;

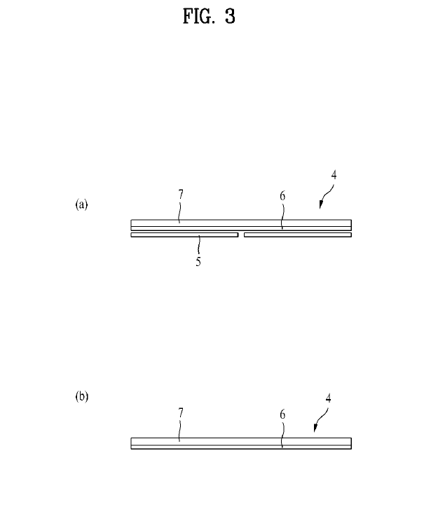

FIG. 4 shows comparative dissolution profile test results for Examples 1 to 6, Comparative Examples 1, 4 and 5 and H; and

FIG. 5 shows transepidermal permeation test results for Examples 1, 4 and 6, Comparative Examples 1 and 4, and H.

### DETAILED DESCRIPTION OF THE INVENTION

Hereinafter, the present invention will be described with reference to the annexed drawings.

As a result of research on a transepidermal drug delivery system composition comprising tulobuterol, the inventors of the present invention prepared a drug layer wherein tulobuterol is physically and chemically completely dissolved using a natural rubber-based adhesive material. The present inventors discovered that a drug layer comprising tulobuterol and a natural rubber-based adhesive material exhibits superior adhesion force and is nontoxic in the body, thus minimizing skin irritation, and exhibits superior drug release and skin permeation comparable to a reference drug, Hokunalin patch (available from Abbott Japan Co. Ltd; hereinafter, referred to as ‘H’). The present invention has been completed, based on the discovery.

In one aspect, the present invention provides a transepidermal drug delivery system comprising: a drug layer comprising tulobuterol and a natural rubber-based adhesive material; and a supporter adhered onto one surface of the drug layer to support the drug layer, wherein the natural rubber-based adhesive material comprises 10 to 40 parts by weight of a natural rubber, 54.5 to 85 parts by weight of a rosin ester resin and an acid value controller, wherein the drug layer has a thickness of 25 μm to 75 μm. As used herein, the term “parts by weight” may be replaced by “wt %”. Specifically, in the case where the total content of ingredients contained in the drug layer is 100 parts by weight, the content of respective ingredients may be represented by “wt %”. In this case, the content of respective ingredients may be represented based on dry weight of the drug layer.

The transepidermal drug delivery system may comprise four layers, i.e., a release film, a drug layer, a supporter and a fixer. Accordingly, in another aspect, the present invention provides a transepidermal drug delivery system comprising: a drug layer comprising tulobuterol and a natural rubber-based adhesive material; a supporter adhered to the bottom of the drug layer, to support the drug layer; a fixer adhered to the bottom of the supporter to suitably fix the drug layer on the skin; and a release film or a release paper which is adhered to the top of the drug layer to protect the drug layer and is readily separated from the drug layer, wherein the natural rubber-based adhesive material comprises 10 parts by weight to 40 parts by weight of a natural rubber, 54.5 to 85 parts by weight of a rosin ester resin and an acid value controller, wherein the drug layer has a thickness of 25 μm to 75 μm. The top and bottom of the drug layer are arbitrarily indicated for better illustration and simply mean different surfaces of the drug layer.

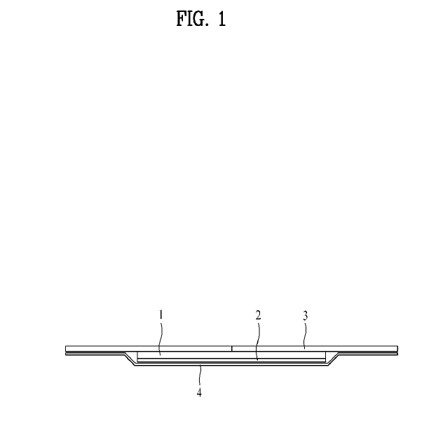

FIG. 1 illustrates a four-layer transepidermal drug delivery system comprising tulobuterol. The four-layer transepidermal drug delivery system exhibits superior drug release and skin permeation effects, comparable to a commercially available reference drug H, and is almost free of skin irritation. In addition, when the transepidermal drug delivery system is adhered to the skin for a long time, the drug layer is firmly fixed to the skin through the fixer, the drug layer is not separated from the skin and the drug is absorbed into the skin at a predetermined rate, thus being useful for treatment of dyspnea of those who suffer from asthma and respiratory diseases.

Referring to FIG. 1, the transepidermal drug delivery system comprises four layers to efficiently permeate the drug into the skin. Specifically, the transepidermal drug delivery system of the present invention may comprise a drug layer 1, a supporter 2, a release film or release paper 3 and a fixer 4.

The drug layer 1 directly contacts the skin and controls drug release and skin permeation rate. The drug layer contains tulobuterol and a natural rubber-based adhesive material. Tulobuterol may be used in a base form or as a salt thereof. Also, tulobuterol is dissolved in the drug layer and the content thereof is not limited to an amount of the drug layer, but may be present in an amount of about 2 mg/10 cm². In addition, the drug layer of the present invention contains no transepidermal absorption accelerator. As used herein, the term “about” means a predetermined range based on a corresponding value (e.g., within ±5%, within ±10%).

The natural rubber-based adhesive material comprises a natural rubber, a rosin ester resin and an acid value controller. The natural rubber controls strength of the drug layer and may be used in an amount of 10 to 40 parts by weight. When the natural rubber content is less than 10 parts by weight, hardness of the drug layer is excessively low, and when the natural rubber content exceeds 40 parts by weight, hardness increases, but adhesiveness is considerably decreased and flexibility is deteriorated. The rosin ester resin acts as a tackifier and is used in an amount of 54.5 to 85 parts by weight. When the rosin ester resin is used in an amount of less than 54.5 parts by weight, adhesiveness is low, making attachment to the skin difficult. In addition, when the rosin ester resin is used in an amount of more than 85 parts by weight or higher, the drug layer is too adhesive and stick residues disadvantageously remain, when separated from the skin. The rosin ester resin comprises rosin glycerin ester, hydrogenated rosin glycerin ester, rosin pentaerythritol ester or a combination thereof. Preferably, the rosin ester resin comprises hydrogenated rosin glycerin ester or a combination thereof.

The rosin ester resin is a rosin derivative, which is composed of abietic acid having a carboxylic group. Accordingly, the rosin ester resin may induce skin irritation due to the carboxylic group and have an acid value. Commercially available rosin ester resins have generally an acid value (mg KOH/g) of 2 to 20. Meanwhile, structurally, tulobuterol has a secondary amine which is chemically bonded to the carboxylic group of the rosin ester resin, thus directly affecting drug dissolution and transepidermal permeation rate. When the affinity between a main ingredient and an adhesive ingradient is excessively high, thermodynamic activity decreases and release of the main ingredient is delayed. In addition, it is most preferred that the rosin ester resin be neutralized with an alkaline material before use, in order to minimize skin irritation caused by the carboxylic group of abietic acid. As such, a considerably essential ingredient acting as a neutralizer to control dissolution and transepidermal permeation rates and reduce skin irritation, the acid value controller, prevents chemical reaction between the carboxylic group of the rosin ester and the secondary amine of tulobuterol.

The acid value controller comprises all pharmaceutically available basic ingredients. The acid value controller includes organic bases such as alkanol amines, alkyl amines, inorganic bases or a combination thereof and is not limited thereto. Alkanol amines include monoethanolamine, diethanolamine, triethanolamine or diisopropanolamine. Preferably, the acid value controller includes triethanolamine. The acid value controller is present in the drug layer in an amount sufficient to neutralize rosin ester. That is, the content of the acid value controller may be within a predetermined range (e.g., within ±5%, within ±10%) based on the neutralization point between the acid value controller and the rosin ester. For example, in the case where triethanolamine is used as the acid value controller and hydrogenated rosin glycerin ester is used, the content of the acid value controller is 0.6 to 2 parts by weight, preferably, 1 to 1.7 parts by weight, but is not limited thereto. The content of the acid value controller depends on factors such as rosin ester content, rosin ester acid value and molecular weight of the acid value controller. Accordingly, the content of the acid value controller may correspond to the content illustrated based on hydrogenated rosin glycerin ester/triethanolamine according to rosin ester and the acid value controller used. Preferably, the content of the acid value controller may be calculated by Equation 1 below:

Content of acid value controller=content of rosin ester×acid value of rosin ester×molecular weight of acid value controller/56100/purity of acid value controller (%)×100 Equation 1

In Equation 1, the acid value of rosin ester means an amount of KOH required to neutralize 1 g of rosin ester (mg KOH/g) and the purity of acid value controller is represented by %.

Examples of the drug layer that can be used in the present invention, if possible, include tocopherol and derivatives thereof, ascorbic acid and derivatives thereof, antioxidants such as dibutylhydroxytoluene (BHT) or butylhydroxyanisol, pigments such as zinc oxide or titanium oxide, preservatives such as propyl paraben, methyl paraben, imidazoline derivatives, pyrimidine derivatives, or dioxane derivatives, and flavors. These additives may be preferably used in an amount of 5 parts by weight or less, more preferably, 2 parts by weight or less, based on the weight of the drug layer.

The drug layer is prepared such that it has a thickness of 25 μm to 75 μm. When the thickness of the drug layer is less than 25 μm, the drug layer cannot be uniformly applied, and application thickness deviation and content deviation are increased. In addition, when the thickness of the drug layer exceeds 75 μm, the concentration of drug contained in the drug layer is low and drug release ratio is thus decreased. In addition, in order to increase the thickness of the drug layer, the concentration of natural rubber-based adhesive material should be relatively increased, but the surface is first dried and pores are thus generated in the drug layer.

The drug layer of the present invention may be prepared by various methods. For example, the drug layer may be prepared by dissolving tulobuterol and a natural rubber-based adhesive material in an organic solvent to obtain a drug layer solution, applying the drug layer solution to a release film, and drying the applied solution. The organic solvent is a mixture of toluene and heptanes wherein, preferably, a ratio of toluene to heptanes is 5˜8:2˜5 (wt/wt or v/v). Under this ratio, tulobuterol and the natural rubber-based adhesive material are readily dissolved. In addition, the drug layer can be dried at a low temperature by controlling an azeotropic point to a low level, and the content of drug can thus be uniformly maintained, as compared to drying at a high temperature.

The supporter 2 supports the drug layer and prevents contamination from foreign materials during patch and prevents reverse diffusion of the drug to permeate the drug into the skin. The supporter comprises a polyester film on which transparent, semi-transparent or non-transparent aluminum is deposited, a polyester film on which a non-woven fabric is laminated, a polyethylene terephthalate film or a combination thereof. Preferably, the supporter has a thickness of 5 μm to 50 μm, more preferably, about 20 μm. When the supporter has a thickness less than 5 μm, it is nonuniformly laminated to the drug layer due to high flexibility in the process of preparation. Meanwhile, when the supporter has a thickness of 50 μm or higher, it may be readily separated from the skin due to excessively low flexibility of the delivery system in spite of high adhesiveness of the drug layer.

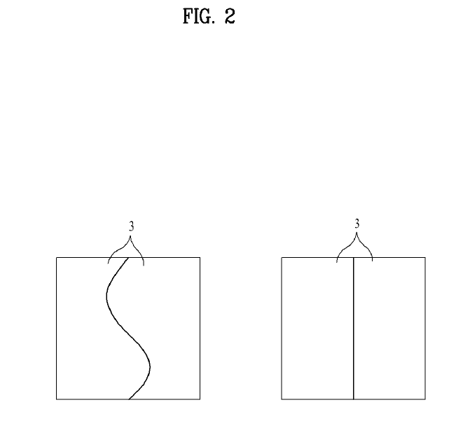

The release film or release paper 3 is adhered to the other surface of the drug layer, protects the drug layer from foreign materials, and is readily separated from the drug layer upon use. The shape of the release film or release paper is shown in FIG. 2. The release film that can be used in the present invention may be a polyester, polyvinylchloride, polyvinylidene chloride, polyethylene terephthalate film. In addition, the release paper may be a paper such as polyolefin-laminated wood free paper or glassine paper. The surface of the release film or release paper contacting the drug layer may be coated with a silicon or fluoride resin so that it is readily separated from the drug layer. Preferably, the release film or release paper exhibits superior stability with passage of time and includes a semi-transparent polyethylene terephthalate release film which has one surface coated with a silicon resin and has a thickness of about 75 μm.

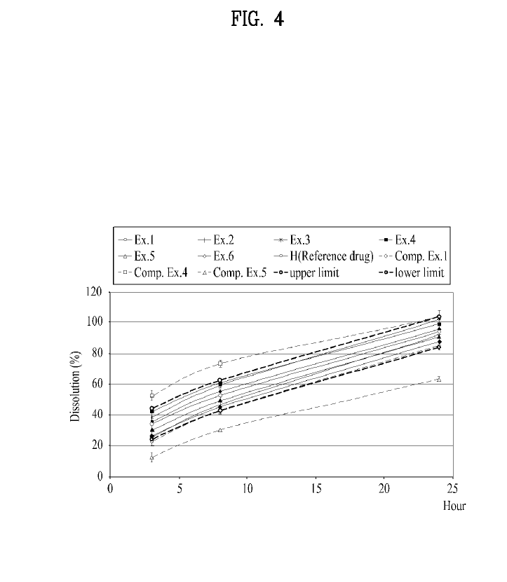

The fixer 4 is adhered to the supporter and fixes the drug layer to the skin for a long time to allow tulobuterol to be uniformly permeated into the skin. That is, the fixer prevents separation of the drug layer from the skin caused by sweat or moisture although patched for a long time, and makes tulobuterol most efficiently permeated into the skin. Examples of the fixer include, but are not limited to, a laminate of a polyethylene terephthalate film and an adhesive-coated polyurethane film (FIG. 4(a)) or an adhesive-coated polyurethane film (FIG. 4(b)). Preferably, the polyurethane film has a thickness of 20 μm to 100 μm. When the polyurethane film has a thickness less than 20 μm, the polyurethane film is readily adhered to the skin, but it cannot be uniformly adhered due to the excessively thin thickness, and when the thickness is 100 μm or higher, the film is excessively thick and thus provides a poor usage sensation and is readily separated from the skin. In addition, the fixer is preferably cut such that it is 5 mm to mm larger than each edge of the drug layer-coated supporter, and may be controlled according to the size and shape of the supporter. Preferably, the fixer includes a laminate with a thickness of about 30 μm of an adhesive-coated polyurethane film and a (cut) polyethylene terephthalate film, which is about 10 mm larger than each edge of the supporter.

### EXAMPLES

Now, the present invention will be described in more detail with reference to the following Examples. These examples are provided only for illustrating the present invention and should not be construed as limiting the scope and spirit of the present invention.

The following Examples and Comparative Examples are selected from various repeated tests performed according to test schemes for better understanding of the present invention. In the following Examples, ingredient addition sequence is not limited to those mentioned in Examples and Comparative Examples.

### Example 1

4.00 wt % of tulobuterol, 30.28 wt % of a natural rubber, 64.46 wt % of hydrogenated rosin glycerin ester and 1.26 wt % of triethanolamine (in which wt % is based on dry weight) were dissolved in a solution of toluene and heptane (7:3, wt/wt). The resulting solution was applied to the surface of a silicon resin-coated semi-transparent polyethylene terephthalate release film with a thickness of 75 μm and dried to prepare a drug layer with a thickness of 50 μm. The drug layer was transcribed to a semi-transparent polyethylene terephthalate film supporter with a thickness of 20 μm, and aged in a thermohydrostat (40±2° C., 75±5%) for 3 days. The drug layer-transcribed supporter was cut to a size of 10 cm² using a mold packer. The cut supporter was laminated to an adhesive-coated fixer and the resulting laminate was cut to a predetermined size.

### Examples 2 to 6 and Comparative Examples 1 to 5

The drug layers of Examples 2 to 6 and Comparative Examples 1 to 5 were prepared under various compositions and thicknesses of drug layer in accordance with the method illustrated in Example 1. Table 1 shows compositions and thicknesses of the drug layer of Examples 2 to 6 and Comparative Examples 1 to 5.

TABLE 1

| | | | | | | Comp | Comp | Comp | Comp | Comp |
|---|---|---|---|---|---|---|---|---|---|---|
| | Ex. 2 | Ex.3 | Ex.4 | Ex.5 | Ex.6 | Ex.1 | Ex.2 | Ex.3 | Ex.4 | Ex.5 |
| Thickness | 50 μm | 50 μm | 25 μm | 60 μm | 75 μm | 50 μm | 50 μm | 50 μm | 20 μm | 80 μm |
| Tulobuterol | 4.00 | 4.00 | 8.00 | 3.33 | 2.67 | 4.00 | 4.00 | 4.00 | 10.00 | 2.50 |
| Natural rubber | 10.00 | 40.00 | 27.99 | 32.67 | 32.03 | 30.00 | 9.33 | 40.50 | 27.50 | 33.76 |
| Hydrogenated | 84.35 | 54.92 | 62.78 | 62.76 | 64.05 | 66.00 | 85.00 | 54.43 | 61.30 | 62.52 |
| rosin glycerin | | | | | | | | | | |
| ester | | | | | | | | | | |
| Triethanolamine | 1.65 | 1.08 | 1.23 | 1.23 | 1.25 | 0.00 | 1.67 | 1.07 | 1.20 | 1.22 |
| Total | 100.00 | 100.00 | 100.00 | 100.00 | 100.00 | 100.00 | 100.00 | 100.00 | 100.00 | 100.00 |

### Experimental Example

In order to evaluate quality of the transepidermal drug delivery systems prepared in Examples 1 and 6 and Comparative Examples 1 to 5 and commercially available Hokunalin patch (H), adhesion force, skin irritation, comparative dissolution profile tests and transepidermal permeation test were performed. In particular, samples used for tensile strength tests of the adhesion force tests, comparative dissolution profile tests and transepidermal permeation tests were not laminated to a fixer in order to compare qualities in the form similar to the commercially available H.

### Experimental Example 1

### Adhesion Force Test

The adhesion force test was carried out by patching the samples of Examples 1 to 6, Comparative Examples 1 to 5 and H onto 20 subjects' backs and evaluating adhesion force after 24 hours in accordance with the measurement scales as set forth in Table 2. In addition, the adhesion force of drug layers of Example 1 and H were compared using a tensile strength tester. The tensile strength test was performed as follows. A sample was cut to a width of 12 mm and then adhered to a phenol resin test plate (width: 25 mm, length: 125 mm, thickness: 5 mm) stood in a thermostat at 37° C. for 30 minutes such that each edge of the sample contacts the edge of the test plate. A rubber roller (850 g) was passed on the sample at a rate of 300 mm/min twice and the sample-adhered test plate was stood in a thermostat at 37° C. for 30 minutes.

The edge of the sample adhered to the test plate was bent back to an angle of 180 degrees, was fixed on an upper part of the tensile strength tester and the test plate was fixed under a lower part thereof. The weight was measured 10 times, when a length of 20 mm was drawn at a rate of 300 mm/min, and an average weight was then obtained.

TABLE 2

| | Very | | | | |
|---|---|---|---|---|---|
| | excellent | Excellent | Medium | Bad | Very bad |
| Measurement | 5.0 | 4.0 | 3.0 | 2.0 | 1.0 |
| scale | | | | | |

### Experimental Example 2

### Skin Irritation Test

Six samples of Examples 1 to 6, Comparative Examples 1 to 5 and H cut in the form of a circle with a diameter of 1.5 cm were adhered to both the antebrachium of 20 subjects for 8 hours. The sample H was fixed with a fixing fabric so that it was not readily separated from the skin. The sample was removed and erythema was observed by the naked eye after 30 minutes in accordance with the measurement scales as set forth in Table 3.

TABLE 3

| | No | Minimal | | Slight | |
|---|---|---|---|---|---|
| | irritation | irritation | Medium | irritation | Irritation |
| | (−−) | (−) | (+−) | (+) | (++) |
| Measurement | 5.0 | 4.0 | 3.0 | 2.0 | 1.0 |
| scale | | | | | |

### Experimental Example 3

### Comparative Dissolution Profile Test

For six samples (10 cm²/one sample) of Examples 1 to 6, Comparative Example 1, Comparative Example 4, Comparative Example 5 and H, the supporter surface was adhered to the center of a disc assembly of a dissolution system with a double-coated tape such that the drug layer was exposed upward. 500 mL of water was charged into a dissolution vessel, and 10 mL of the dissolution solution was collected at a dissolution temperature of 32±0.5° C., at a paddle rotation rate of 50 rpm at 3, 8 and 24 hours. In accordance with a general pharmacopoeia absorbance measurement test, absorbance was measured at a wavelength of 211 nm. Similarity tests were performed in accordance with a paddle over disk method of United States Pharmacopeia (USP) transepidermal drug delivery system.

### Experimental Example 4

### Transepidermal Permeation Test

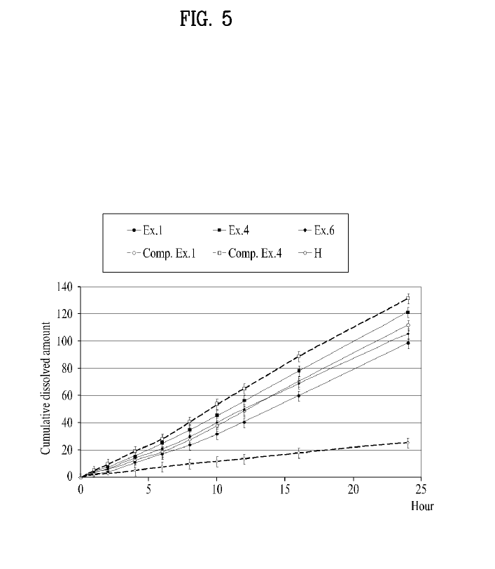

In Comparative dissolution profile tests, Examples 1, 4 and 6 among Experimental Examples which exhibit dissolution behavior equivalent to H used as a reference drug within an acceptable deviation range (±10%) were selected and transepidermal permeation tests were performed for Comparative Examples 1 and 4 and the reference drug H. 6-week old male hairless mice were cervically dislocated, a 3 cm×3 cm patch of skin was enucleated and subcutaneous fat was removed. Samples of Examples 1, 4 and 6, Comparative Examples 1 and 4, and H which were previously cut in the form of a circle with a size of 1.767 cm² were adhered to the epidermal layer of the enucleated skin. The endodermal layer of the skin was adhered such that it laid toward Franz diffusion cells. A receptor layer of Franz diffusion cells was carefully charged with a phosphate buffered saline (PBS) buffer solution (pH 7.4) such that foams were not generated, and the resulting mixture was stirred at 600 rpm, while a Franz diffuser temperature was maintained at 32±0.5° C. 250 μl of the samples were collected at 0, 1, 2, 4, 6, 8, 10, 12, 16 and 24 hours, the amount of transepidermally permeated tulobuterol was assayed by HPLC and an equivalent amount of buffer solution was charged in the receptor layer. An active skin area of the hairless mice adhered to the Franz diffusion cells was 1.767 cm², a volume of the receptor was 13.0 mL, and skin permeation tests were performed under sink conditions. Under HPLC assay conditions, a mixture of 0.02M potassium dihydrogen phosphate and acetonitrile (80:20, v/v) was used as a mobile phase, an injection amount was 20 μl, a flow rate was 1.2 mL/min, a detection wavelength was 211 nm, and a column was Agilent Zorbox XDB C18. The transepidermal permeation test results are shown in FIG. 5.

Test Results

The adhesion force test results are shown in Table 4. The adhesion force is an essential physical property for drug absorption passage. The samples of Examples 1 to 6 and Comparative Examples 1 to 5 were patched, while maintaining the initial state, until the product samples were intentionally separated from all subjects (24 hours) and exhibited superior adhesion force, as compared to the reference drug (H). The sample of Comparative Example 2 was suitably patched on the skin even after 24 hours, but a large amount of adhesive remained on the skin, even after separation. In addition, as a result of tests using a tensile strength tester, adhesion force of the drug layers of Example 1 and H was 597.98±79.97 gf and 359.91±37.88 gf, respectively. The drug layer of Example 1 exhibited superior adhesion force, as compared to the reference drug H. The reason why the samples in accordance with Examples of the present invention exhibited superior adhesion force is that the natural rubber-based adhesive material contained in the drug layer exhibited superior adhesion force owing to a suitable composition of the natural rubber and the rosin ester resin used as an adhesion-imparting agent, in particular, the drug layer is uniformly fixed to the skin through a fixer.

TABLE 4

| Experimental | Examples | | | | | | Comparative Examples | | | | | |
|---|---|---|---|---|---|---|---|---|---|---|---|---|
| Examples | 1 | 2 | 3 | 4 | 5 | 6 | 1 | 2 | 3 | 4 | 5 | H |
| Adhesion force | 5.0 | 5.0 | 5.0 | 5.0 | 5.0 | 5.0 | 5.0 | 5.0 | 5.0 | 5.0 | 5.0 | 3.6 |

Skin irritation test results are shown in Table 5. Examples 1 to 6 and Comparative Examples 3 and 5 were known to be almost free of skin irritation. The reference drug H exhibited a medium level of irritation, and Comparative Examples 1 and 4 exhibited serious irritation, as compared to the reference drug. Comparative Example 1 contains no acid value controller and so carboxyl groups contained in an adhesion-imparting resin seemed to induce skin irritation. In addition, in Comparative Example 4, the content of the drug higher than other ingredients was believed to directly cause skin irritation. In Comparative Example 2, 9 subjects underwent erythema, while the adhesive left on the skin was removed with an alcohol gauze.

TABLE 5

| Experimental | Examples | | | | | | Comparative Examples | | | | | |
|---|---|---|---|---|---|---|---|---|---|---|---|---|
| Examples | 1 | 2 | 3 | 4 | 5 | 6 | 1 | 2 | 3 | 4 | 5 | H |
| Average | 4.90 | 4.90 | 4.90 | 4.80 | 4.90 | 4.92 | 2.84 | — | 4.90 | 3.14 | 4.95 | 3.22 |

The comparative dissolution profile test and the transepidermal permeation test are good methods to evaluate equivalence to the reference drug with respect to efficacies and effects of the present invention. In particular, the comparative dissolution profile test is considerably important for identifying biological equivalence to the reference drug, and biological equivalence was accepted, when an acceptable dissolution range between the drug of the present invention and the reference drug is ±10%. The comparative dissolution profile test was carried out by comparing the reference drug H with Examples 1 to 6, and Comparative Examples 1, 4 and 5 except Comparative Example 2, wherein the adhesive disadvantageously remains on the skin, and Comparative Example 3 wherein adhesion force was excessively low. The test results are shown in FIG. 4. In the comparative dissolution profile test, Examples 1 to 6 correspond to ±10%, an acceptable deviation range with the reference drug H. Comparative Example 1 wherein the drug layer contains no acid value controller exhibited a dissolution rate substantially equivalent to a lower limit. Comparative Example 4, wherein the drug layer has a small thickness and a high drug concentration, was out of an acceptable dissolution deviation upper limit.

The transepidermal permeation tests were carried out by selecting Examples 1, 4 and 6 which exhibited equivalent dissolution behaviors within an acceptable deviation range (±10%) with the reference drug H in Experimental Examples, and comparing Comparative Examples 1 and 4 with the reference drug H with respect to transepidermal permeation behaviors. The results thus obtained are shown in FIG. 5. Comparative Example 1, wherein the drug layer contained no acid value controller, released the drug at a constant rate even with passage of time. Drug release is delayed due to chemical bonds between the adhesion-imparting resin having a carboxyl group and secondary amine of the tulobuterol in the drug layer. In addition, Comparative Example 4, wherein a drug was present in a high amount, exhibited high drug release as compared to other compositions, and Examples 1, 4 and 6 exhibited drug release behaviors in the range equivalent to the reference drug H.

As apparent from the afore-going, the present invention provides a transepidermal drug delivery system containing tulobuterol and a natural rubber-based adhesive material. In addition, the present invention provides a transepidermal drug delivery system comprising four layers, i.e., the drug layer, the release film or the release paper and the fixer. The transepidermal drug delivery system exhibits efficient drug release and skin permeation effects, comparable to a commercially available reference drug. In addition, the drug layer which directly contacts the skin is treated with ingredients harmless to the body and is almost free of skin irritation. In addition, since the drug layer is uniformly fixed to the skin through the fixer, it is not separated from the skin and is thus absorbed into the skin at a predetermined rate, although it is adhered to the skin for a long time. Accordingly, the transepidermal drug delivery system is useful for treating dyspnea of patients who suffer from asthma and respiratory diseases.

The present invention may be applied to pharmaceutical compositions containing tulobuterol, specifically, transepidermal drug delivery systems containing tulobuterol.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A transepidermal drug delivery system comprising:
a drug layer containing tulobuterol and a natural rubber-based adhesive material; and
a supporter adhered to one surface of the drug layer to support the drug layer,
wherein the natural rubber-based adhesive material comprises 10 to 40 parts by weight of a natural rubber, 54.5 to 85 parts by weight of a rosin ester resin and an acid value controller, and the drug layer has a thickness of 25 μm to 75 μm.

2. The transepidermal drug delivery system according to claim 1, wherein the acid value controller comprises a pharmaceutically available organic or inorganic base, or a combination thereof.

3. The transepidermal drug delivery system according to claim 2, wherein the acid value controller is obtained in accordance with Equation 1 below:
Content of acid value controller=content of rosin ester×acid value of rosin ester (mg KOH/g)×molecular weight of acid value controller/56100/purity of acid value controller (%)×100 (1)

4. The transepidermal drug delivery system according to claim 1, wherein the rosin ester resin comprises rosin glycerin ester, hydrogenated rosin glycerin ester, rosin pentaerythritol ester or a combination thereof.

5. A transepidermal drug delivery system comprising:
a drug layer containing tulobuterol and a natural rubber-based adhesive material;
a supporter adhered to one surface of the drug layer to support the drug layer;
a fixer adhered to the bottom of the supporter to suitably fix the drug layer to the skin; and
a release film or a release paper adhered to the top of the drug layer to protect the drug layer, the release film or release paper being readily separated from the drug layer,
wherein the natural rubber-based adhesive material comprises 10 to 40 parts by weight of a natural rubber, 54.5 to 85 parts by weight of a rosin ester resin and an acid value controller, and the drug layer has a thickness of 25 μm to 75 μm.

6. The transepidermal drug delivery system according to claim 5, wherein the acid value controller comprises a pharmaceutically available organic or inorganic base, or a combination thereof.

7. The transepidermal drug delivery system according to claim 6, wherein the acid value controller is obtained in accordance with Equation 1 below:
Content of acid value controller=content of rosin ester×acid value of rosin ester (mg KOH/g)×molecular weight of acid value controller/56100/purity of acid value controller (%)×100 (1)

8. The transepidermal drug delivery system according to claim 5, wherein the rosin ester resin comprises rosin glycerin ester, hydrogenated rosin glycerin ester, rosin pentaerythritol ester or a combination thereof.

9. The transepidermal drug delivery system according to claim 5, wherein the fixer is an adhesive-coated polyurethane film or a laminate of a cut polyethylene terephthalate film and the adhesive-coated polyurethane film,
wherein the polyurethane film has a thickness of 20 μm to 100 μm.

10. The transepidermal drug delivery system according to claim 9, wherein the fixer is cut such that it is 5 to 20 mm larger than each edge of the supporter.
